(19) United States
(12) Patent Application Publication
(10) Pub. No.: US 2024/0099944 A1
(43) Pub. Date: Mar. 28, 2024

(54) Stable Skin Care Compositions Containing a Retinoid

(71) Applicant: The Procter & Gamble Company, Cincinnati, OH (US)

(72) Inventors: Claire Noel Wallace, Cincinnati, OH (US); Joseph Harry Jansen, Harrison, OH (US); Kai Lin Florence Ng, Singapore (SG)

(21) Appl. No.: 18/460,804

(22) Filed: Sep. 5, 2023

Related U.S. Application Data
(60) Provisional application No. 63/410,762, filed on Sep. 28, 2022.

(51) Int. Cl. A61K 8/06; A61K 8/23; A61K 8/25; A61K 8/35; A61K 8/64; A61K 8/67; A61Q 19/00

(52) U.S. Cl. CPC A61K 8/062; A61K 8/23; A61K 8/25; A61K 8/35; A61K 8/64; A61K 8/671; A61Q 19/00; A61K 2800/30; A61K 2800/51; A61K 2800/522; A61K 2800/524

(57) ABSTRACT

A skin care emulsion composition that includes a continuous phase and a dispersed phase containing one or more retinoids. The continuous phase has a preservative system that includes one or more cell wall disrupters. The preservative system can be free of DMDM hydantoin, imadazolidinyl urea, diazlidinyl urea, sodium hydroxyl, methyl glycinate. The continuous phase can also include one or more antioxidants and one or more reducing agents. The composition can be chemically and physically stable.

### FIELD OF THE INVENTION

The present invention generally relates to a stable skin care composition containing a retinoid. More specifically, the present invention relates to a skin care composition in the form of an oil-in-water emulsion containing a stable retinoid.

### BACKGROUND OF THE INVENTION

Skin care products can improve the health and/or appearance of a user's skin. Currently, there are a variety of topical skin care products available that are directed to delaying, minimizing, or even eliminating skin wrinkling and other histological changes typically associated with the aging of skin and/or environmental damage to human skin. For at least some people, fine lines, wrinkles, and discoloration in the skin are a reminder of the disappearance of youth. As a result, the elimination of wrinkles and discoloration has become a booming business in youth-conscious societies. Treatments range from cosmetic moisturizers and serums to various forms of cosmetic surgery.

Many skin care products, including moisturizers and serums, can include vitamins, vitamin derivatives, and/or other active ingredients for improving skin appearance. For example, vitamin A, also referred to as retinol, is known for use in topical skin care compositions to provide skin health and/or appearance benefits. Vitamin A, along with its derivatives, form a class of compounds commonly referred to as “retinoids.” At one time, retinoids were primarily used for the treatment of acne. More recently, retinoids have also been used in the treatment of photo- and/or intrinsically aged skin.

However, formulating stable skin care products containing a retinoid with a three-year shelf life can be challenging because retinoids are undesirably reactive and susceptible to degradation. Some known sources of degradation include oxidation, light exposure (e.g., ultraviolet radiation), heat (e.g., temperatures of 40° C. or more), and interactions with other ingredients in the formula. As retinoids degrade, they are less efficacious and the composition can yellow and it can have a noticeable odor, reminiscent of sour milk.

Furthermore, there is pressure to reformulate some retinoid products because some consumers want skin care products that have cleaner ingredient lists, including formulas without formaldehyde-releasing agents. For example, DMDM hydantoin (commercially available as Glydant®) is a common effective preservative for skin care products, including stable retinoid products. However, since it is a formaldehyde-releasing agent, it can be desirable to remove it from skin care products. However, it is difficult to identify a preservative system that is both effective and does not impact retinoid stability.

Accordingly, it would be desirable to provide a retinoid-containing skin care composition with a preservative system that is free from formaldehyde-releasing agents where the color and retinoid concentration are stable.

### SUMMARY OF THE INVENTION

A skin care composition comprising: (a) a continuous phase comprising: (i) a preservative system comprising hydroxyacetophenone; (ii) carnosine; (iii) sodium sulfite; (iv) water; (b) a dispersed phase comprising retinyl propionate; wherein the composition is free of DMDM hydantoin, imadazolidinyl urea, diazlidinyl urea, sodium hydroxyl, and methyl glycinate.

A skin care composition comprising: (a) a continuous phase comprising: (i) from about 0.1% to about 1% of a preservative system comprising one or more cell wall disrupters; (ii) from about 0.01% to about 1% of one or more antioxidants; (iii) from about 0.02% to about 0.25% of one or more reducing agents; (iv) water; (b) a dispersed phase comprising from about 0.01% to about 2% of one or more retinoid; wherein the skin care composition is free of formaldehyde and formaldehyde-releasing agents.

### BRIEF DESCRIPTION OF THE DRAWINGS

The patent or application file contains at least one drawing executed in color. Copies of this patent or patent application publication with color drawings will be provided by the Office upon request and payment of the necessary fee.

While the specification concludes with claims particularly pointing out and distinctly claiming the subject matter of the present invention, it is believed that the invention can be more readily understood from the following description taken in connection with the accompanying drawings, in which:

FIG. 1 shows the % retinyl propionate (RP) remaining vs. time for Examples A-G;

FIG. 2A is a photograph of Example 2, which had 54% RP remaining and a b-value of 16.32 after storage under accelerated stability conditions;

FIG. 2B shows an Example 4, which had 63% RP remaining and a b-value of 17.04 after storage under accelerated stability conditions;

FIG. 3A shows the % RP remaining for Examples 1, 2, and 3;

FIG. 3B shows the yellow discoloration as determined by a b-value for Examples 1, 2, and 3;

FIG. 4A shows Serum Example 5 after storage under accelerated stability conditions; and

FIG. 4B shows Serum Example 6 after storage under accelerated stability conditions.

### DETAILED DESCRIPTION OF THE INVENTION

Retinoids are a class of compounds derived from Vitamin A that are frequently incorporated into skin care compositions for anti-wrinkle, acne, and hyperpigmentation benefits. It can be challenging to formulate with retinoids because they have a high propensity for degradation. Retinoids are sensitive to oxidation, hydrolysis, heat, and UV. As retinoids degrade, there is less active available to penetrate the skin and the skin care product becomes less effective. In addition, the formula can be noticeably more yellow (as measured by the b-value in the Color Test Method, described hereafter) and can have an off-putting smell, reminiscent of sour milk.

DMDM hydantoin (commercially available as Glydant®) and DMDM hydantoin and iodopropynyl butylcarbamate (commercially available as Glydant® Plus Liquid) are common preservatives in skin care products and can be used as the preservative in stable retinoid formulations. However, DMDM hydantoin is a formaldehyde-releasing agent and some consumers and regulators would prefer products that are free of formaldehyde-releasing agents.

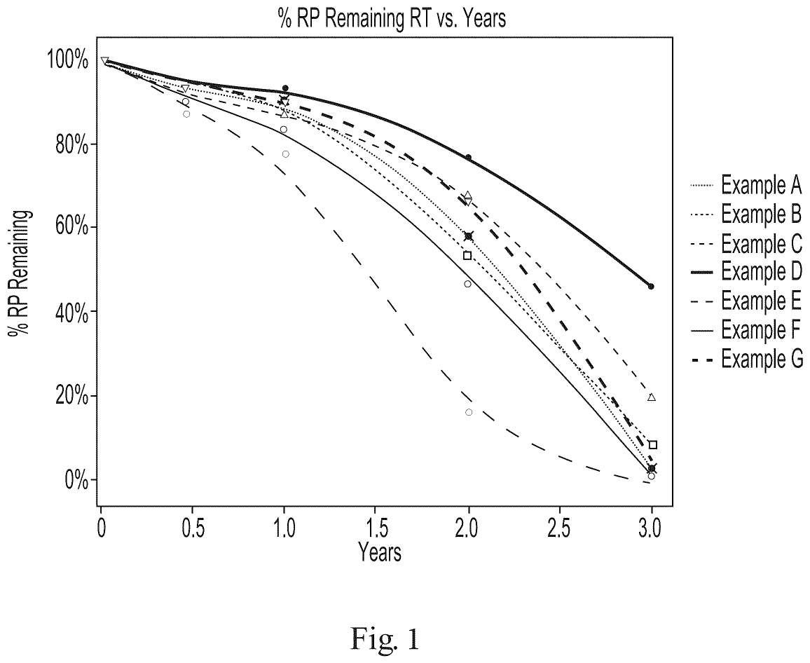

However, identifying a replacement preservative that is both effective at inhibiting microbial growth and does not interact with retinoids was difficult. FIG. 1, shows the % retinyl propionate (RP) remaining vs. time for Examples A-G (see Table 2, hereafter), which comprise Chassis 1 (see Table 1, hereafter) with the addition of various preservative systems or Chassis 2. The % RP remaining was determined by the HPLC Test Method, described herein. The Examples were stored at ambient conditions in a container that limits the free flow of oxygen (e.g., an aluminum tube, laminated tube, glass jar, HDPE jar, PP jar, HDPE pump) away from direct light for three years.

TABLE 1
Chassis 1 and 2

| | Chassis 1 | Chassis 2 |
|---|---|---|
| | (Wt. %) | (Wt. %) |
| Water Phase | | |
| Water | Q.S. | Q.S. |
| Humectant | 3 | 3 |
| Polyacrylamide, C13-14 isoparaffin, and laureth-7 | 2 | 2 |
| (Sepigel 305 ™ from Seppic ®) | | |
| Disodium EDTA | 0.05 | 0 |
| Oil Phase | | |
| Silicone Oils | 22 | 22 |
| Laureth-4 | 0.2 | 0.2 |
| Polysorbate 20 | 0.2 | 0.2 |
| Retinyl Propionate | 0.3 | 0.3 |

TABLE 2

| Ex. | Chassis | Preservative System |
|---|---|---|
| A | Chassis 1 | 0.05% Benzyl Alcohol |
| | | 0.15% Hydroxyacetophenone (Symsave ® H from |
| | | Symrise ®) |
| B | Chassis 1 | 0.05% Phenoxyethanol |
| | | 0.15% Hydroxyacetophenone (Symsave ® H from |
| | | Symrise ®) |
| C | Chassis 1 | 0.2% Benzyl Alcohol |
| | | 0.25% Phenoxyethanol |
| | | 0.05% Sodium Benzoate |
| D | Chassis 1 | 0.3% DMDM hydantoin and iodopropynyl |
| | | butylcarbamate (Glydant ® Plus Liquid) |
| E | Chassis 1 | 0.5% Benzyl Alcohol |
| | | 0.1% Methylparaben |
| F | Chassis 1 | 0.5% 1,2-Hexanediol (and) Caprylyl Glycol (Symdiol ® |
| | | 68 from Symrise) |
| | | 0.375% Phenoxyethanol |
| G | Chassis 2 | None |

FIG. 1 shows Example D, which has a preservative system containing DMDM hydantoin and iodopropynyl butylcarbamate (Glydant® Plus Liquid). This preservative system is known to effectively inhibit microbes and it has the least amount of retinyl propionate degradation at the end of the three-year period, as compared to the other compositions that were tested. Current retinoid products with this preservative system can have a three-year shelf life. However, some consumers would prefer products without this preservative system because DMDM hydantoin is a formaldehyde-releasing agent.

FIG. 1 shows that none of the other preservative systems shown in Examples A-C and E-F have sufficient RP remaining after three-years to be shelf stable for three years and are therefore not satisfactory. In fact, many of these formulas had less than 20% RP remaining at three years. Since the preservative system in these examples is free of formaldehyde-releasing agents and was an effective antimicrobial, more studies were performed to determine if the RP could be stabilized another way. It was determined that hydroxyacetophenone (commercially available as Symsave® H from Symrise®) was a good starting point for further exploration.

Table 3, hereafter, shows the % RP remaining for an experimental skin care composition, Example 2 (see Table 5, hereafter, for the formula for a skin care composition containing RP and a preservative system comprising 0.05% phenoxyethanol and 0.15% hydroxyacetophenone) with the addition of the antioxidant as compared to Example 2 without the antioxidant. This test was performed under accelerated stability conditions of 40° C. and 75% relative humidity for 6 months away from direct light and the samples were stored in containers that limited the free flow of oxygen. The % RP was determined by the HPLC Method, described hereafter.

TABLE 3

| | | % RP |
|---|---|---|
| | | Remaining |
| | | vs. Example 2 |
| | Antioxidant | (control) |
| Example 2 | 2.0% PEG-4 (and) Hydroxycinnamic Acid | −13% |
| | containing 0.29% hydroxycinnamic acid | |
| | (commercially available as Lipobrite ™ | |
| | HCA-4) | |
| Example 2 | 0.5% Ferulic Acid | −8% |
| Example 2 | 0.1% Bakuchiol (Sytenol ® A) | −6% |
| Example 2 | 0.1% Camellia sinensis Leaf Extract | −3% |
| | (Crodarom ® White Tea EC) | |
| Example 2 | 0.5% Caffeine | 2% |
| Example 2 | 0.1% Caffeine | 3% |
| Example 2 | 0.05% Rosmarinus officinalis (Rosemary) | 5% |
| | Leaf Extract (NATPURE ® XTRA | |
| | VITALITY from Sensient ®) | |
| Example 2 | 0.1% Rosmarinus officinalis (Rosemary) | 5% |
| | Leaf Extract (from Bell Flavor and | |
| | Fragrances) | |
| Example 2 | 0.2% Rosmarinus officinalis (Rosemary) | 6% |
| | Leaf Extract (from Bell Flavor and | |
| | Fragrances) | |
| Example 2 | 0.1% Ferulic Acid | 9% |
| Example 2 | 0.1% Octadecyl Di-t-butyl-4- | 10% |
| | hydroxyhydrocinnamate | |
| Example 2 | 0.1% Carnosine | 12% |
| Example 2 | 0.1% Ethylene bis(oxyethylene) bis-(3-(5- | 12% |
| | tert-butyl-4-hydroxy-m-tolyl)propionate) | |
| Example 2 | 0.5% Carnosine | 13% |
| Example 2 | 0.05% Punica granatum Fruit Extract | 14% |
| Example 2 | 0.5% Ethylene bis(oxyethylene) bis-(3-(5- | 14% |
| | tert-butyl-4-hydroxy-m-tolyl)propionate) | |
| Example 2 | 0.1% Butylated hydroxytoluene | 15% |
| Example 1 | | 15% |
| (see Table 5) | | |

Although there were several antioxidants that performed well and improved RP stability, carnosine was ultimately selected to move forward because it is a di-peptide and may be consumer preferred for skin care compositions, as compared to other tested antioxidants.

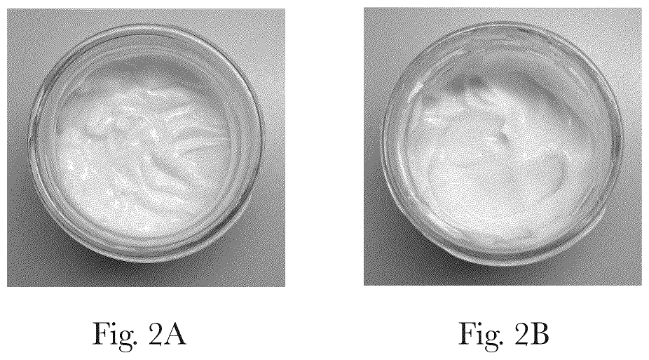

It is known that as retinoids degrade, the skin care composition yellows. This color change can be consumer noticeable, and many consumers believe that yellowing means the retinoid is less effective. Surprisingly, it was discovered that even though RP was relatively stable with carnosine, the product still yellowed under accelerated stability conditions (40° C., 75% RH for 6 months). FIG. 2A shows Example 2 (see Table 5, hereafter), which had 54% RP remaining and a b-value of 16.32 under accelerated stability conditions (40° C., 75% RH for 6 months). FIG. 2B shows an example with Example 2 (see Table 5, hereafter) and 0.1% carnosine. The Example in FIG. 2B had 63% RP remaining and a b-value of 17.04 under accelerated stability conditions (40° C., 75% RH for 6 months). It was surprising that the formula with more stable RP had a higher b-value and was yellower. This yellowing is not consumer acceptable because consumers believe that yellower products are less effective and/or adulterated.

Therefore, additional ingredients needed to be tested to reduce the yellowing. Table 4, hereafter, compares the b-value for Example 2 (see Table 5, hereafter) with an additional ingredient compared to Example 2. This test was performed under accelerated stability conditions of 60° C., away from direct light for two weeks and the samples were stored in containers that limited the free flow of oxygen. The examples where the b-value vs. the control were noticeably less yellow by a human viewer with the unaided eye (except for standard corrective lenses adapted to compensate for near-sightedness, farsightedness, or stigmatism, or other corrected vision) in lighting at least equal to the illumination of a standard 100-watt incandescent white light bulb at 30 cm. The b-value was determined by the Color Test Method, described hereafter.

As shown in Table 4, sodium sulfite, a reducing agent, was the only ingredient tested that dramatically reduced the composition's b-value and the compositions with both 0.05% and 0.1% sodium sulfite could be noticeably less yellow.

TABLE 4

| | | b-value vs. Example 2 |
|---|---|---|
| | Additional Ingredient | (Control) |
| Example 2 | 0.05% Punica granatum Fruit | −6.87 |
| | Extract | |
| Example 2 | 0.1% Ferulic Acid | −4.12 |
| Example 2 | 0.5% Ethylene bis(oxyethylene) | −3.91 |
| | bis-(3-(5-tert-butyl-4-hydroxy-m- | |
| | tolyl)propionate) | |
| Example 2 | 0.1% Camellia sinensis Leaf | −2.12 |
| | Extract (Crodarom ® White Tea | |
| | EC) | |
| Example 2 | 0.1% Rosmarinus officinalis | −1.87 |
| | (Rosemary) Leaf Extract (from | |
| | Bell Flavor and Fragrances) | |
| Example 2 | 0.1% Bakuchiol (Sytenol ® A) | −1.64 |
| Example 2 | 0.1% Rosmarinus officinalis | −1.56 |
| | (Rosemary) Leaf Extract (from | |
| | Bell Flavor and Fragrances) | |
| Example 2 | 0.1% Caffeine | −0.98 |
| Example 2 | 0.1% BHT | −0.09 |
| Example 2 | 0.5% Caffeine | −0.05 |
| Example 2 | 0.1% Ethylene bis(oxyethylene) | 0.08 |
| | bis-(3-(5-tert-butyl-4-hydroxy-m- | |
| | tolyl)propionate) | |
| Example 2 | 0.5% Carnosine | 0.25 |
| Example 2 | 0.1% Carnosine | 0.51 |
| Example 2 | 0.05% Sodium Sulfite | 2.94 |
| Example 2 | 0.1% Sodium Sulfite | 3.83 |

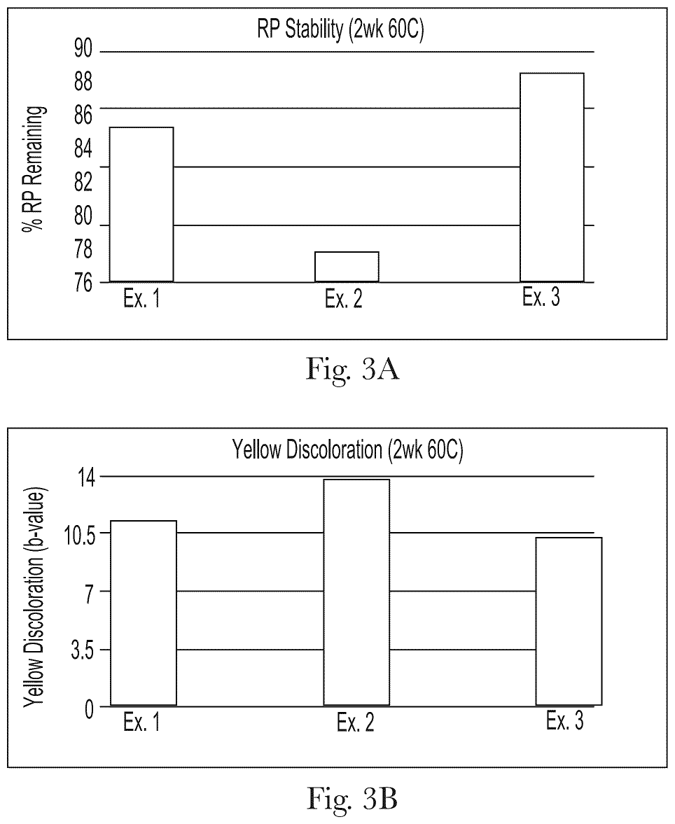

FIGS. 3A and 3B show the stability and discoloration under accelerated stability conditions of 60° C. for two weeks for Examples 1, 2, and 3, as shown Table 5, hereafter. Example 1 is similar to a currently available retinoid formulation with a preservative system containing DMDM hydantoin and iodopropynyl butylcarbamate (commercially available as Glydant® Plus Liquid). In Example 2, the DMDM hydantoin and iodopropynyl butylcarbamate preservative is replaced with a preservative system comprising hydroxyacetophenone and phenoxyethanol. Example 3 has a preservative system hydroxyacetophenone and phenoxyethanol, 0.1% carnosine, and 0.05% sodium sulfite. FIG. 3A shows that the % RP remaining for Example 3 is higher than Examples 1 and 2. FIG. 3B shows that Example 3 has a lower b-value and is therefore less yellow, as compared to Examples 1 and 2. Therefore, Example 3 may be more physically and chemically stable and may be consumer preferred as compared to formulas like Examples 1 and 2.

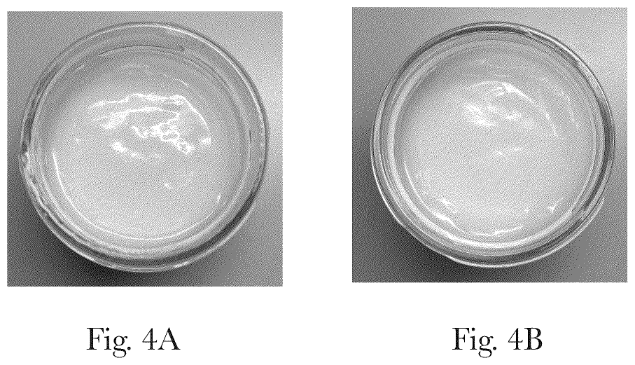

FIGS. 4A and 4B show serum formulas containing retinoids after two weeks at accelerated stability conditions of 60° C. where the samples were stored away from direct light in containers that limited the free flow of oxygen. Serums are believed to behave similarly to moisturizers, in terms of physical and chemical stability of retinoids. Example 5 (see Table 6) has a preservative system that includes hydroxyacetophenone and phenoxyethanol and Example 6 has the same preservative system as Example 5 and also includes 0.1% carnosine and 0.05% sodium sulfite. The composition in FIG. 4B is significantly whiter with a b-value of 2.42, as compared to the serum composition in FIG. 4A with a b-value of 14.86. Thus, Example 6 may be consumer preferred.

### Definitions

As used herein, “formulated without” means that the ingredient is not intentionally added. However, “formulated without” does not guarantee “100% free from” since trace contaminants are possible.

As used herein, “skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin; preventing damage to skin via antioxidant approaches, including UV A and UV B induced damage, preventing formation of comedomes, balancing the skin microbiome or preventing acne.

As used herein, “skin care active” means a compound or combination of compounds that, when applied to skin, provide an acute and/or chronic benefit to skin or a type of cell commonly found therein. Skin care actives may regulate and/or improve skin or its associated cells (e.g., improve skin elasticity, hydration, skin barrier function, and/or cell metabolism). In some examples, the skin care active comprises one or more retinoids.

As used herein, “skin care composition” means a composition that includes a skin care active and regulates and/or improves skin condition.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### Composition

The skin care composition can be a homogenous emulsion that includes a continuous phase and a dispersed phase. The continuous phase can include a dermatologically acceptable carrier, which can typically include water, polymeric thickening agents, antioxidants, and the preservative system. The dispersed phase can include oils, including silicone oils and retinoids.

The dermatologically acceptable carrier enables other components (e.g., actives) to be delivered to the skin at an appropriate concentration. The carrier can thus act as a diluent, dispersant, solvent, or the like for particulate material, which helps ensure that it can be applied to and distributed evenly over the selected target at an appropriate concentration. The carrier may contain one or more dermatologically acceptable solid, semi-solid or liquid fillers, diluents, solvents, extenders and the like. The carrier may be solid, semi-solid, or liquid. In some instances, the carrier can be inert or it can provide benefits of its own to keratinous tissue. Concentrations of the carrier can vary with the carrier selected and the intended concentrations of the composition components.

The type of carrier utilized in the present skin care composition depends on the type of product form desired for the composition. The topical composition useful in the subject invention may be made into a wide variety of product forms such as are known in the art. These include, but are not limited to, lotions, creams, gels, sticks, sprays, ointments, pastes, mousses and cosmetics (e.g., solid, semi-solid, or liquid make-up, including foundations, eye-makeup, pigmented or non-pigmented lip treatments, e.g., lipsticks, and the like). These product forms may comprise several types of carriers including, but not limited to, solutions, aerosols, emulsions, gels, solids, and liposomes.

The skin care composition can be free of or formulated without formaldehyde and formaldehyde-releasing agents. The skin care composition can be free of DMDM hydantoin, imadazolidinyl urea, diazlidinyl urea, sodium hydroxyl, methyl glycinate, and combinations thereof. The skin care composition can also be free of parabens and/or sulfate-based surfactants including SLS and SLES.

The composition can have a b-value of less than 25, less than 20, less than 18, less than 14, alternatively less than 13, alternatively less than 12, alternatively less than 11, and alternatively less than or equal to 10 according to the Test Method, described hereafter.

### Continuous Phase/Water Phase

### Preservative System

As used herein, a preservative system can be one or more substances and/or chemicals that is added to cosmetic compositions that can prevent decomposition by microbial growth and/or by undesirable chemical changes. The preservative system can be free of formaldehyde and formaldehyde-releasing agents. In some examples, the preservative system can contain one or more ingredients that are registered as preservatives by the European Chemicals Agency as of Sep. 27, 2022 (hereinafter ECA). In other examples, the preservative system can contain one or more ingredients that are not registered as preservatives by the ECA. Alternatively, the preservative system can be free of ingredients that are registered as preservatives by the ECA.

The composition can contain from about 0.01% to about 1.5%, alternatively from about 0.05% to about 1.25%, alternatively from about 0.1% to about 1% of the preservative system, alternatively from about 0.2% to about 0.8%, and alternatively from about 0.3% to about 0.5%.

The preservative system can include one or more cell wall disrupters, which can inhibit microbial growth by disrupting microbial cell walls. Non-limiting examples of cell wall disrupters can include aromatic alcohols, mid chain diols, organic acids, hydroxyacetophenone, 1,2-Hexanediol (and) Caprylyl Glycol, ethylhexylglycerin, methylheptylglycerin, or a combination thereof.

Non-limiting examples of aromatic alcohols can include phenoxyethanol, tryptophol, tyrosol, phenethyl alcohol, benzyl alcohol, or a combination thereof.

Non-limiting examples of mid chain diols can include caprylyl glycol, pentylene glycol, hexylene glycol, or a combination thereof.

Non-limiting examples of organic acids can include benzoic acid, sorbic acid, levulinic acid, and salicylic acid, or a combination thereof.

### Chelating Agent

The cosmetic compositions can include from about 0.05% to about 0.5% chelating agent, and alternatively from about 0.1% to about 0.3%. As used herein, “chelating agent” means an active agent capable of removing a metal ion from a system by forming a complex so that the metal ion cannot readily participate in or catalyze chemical reactions. In addition, chelating agents can be used in combination with the preservative system described herein to boost the efficacy of the preservative. Non-limiting examples of chelating agents can include EDTA (disodium EDTA, tetrasodium EDTA), tetrahydroxypropyl ethylenediamine, etidronic acid, sodium phytate, phytic acid, oxalic acid and derivatives, sodium gluconate, EDDS (trisodium ethylenediamine disuccinate), or a combination thereof.

### Antioxidant

The cosmetic compositions can include from about 0.001% to about 1%, alternatively from about 0.005% to about 0.85%, alternatively from about 0.01% to about 0.75% antioxidant, alternatively 0.05% to about 0.5%, and alternatively from about 0.1% to about 0.3%.

The antioxidant can be a non-vitamin antioxidant. Non-limiting examples of antioxidants can include hydroxycinnamic acid, ferulic acid, bakuchiol, tea extracts including Camellia sinensis leaf extract (white tea) and green tea extract, caffeine, Rosmarinus officinalis leaf extract, Octadecyl Di-t-butyl-4-hydroxyhydrocinnamate, carnosine, Ethylene bis(oxyethylene) bis-(3-(5-tert-butyl-4-hydroxy-m-tolyl)propionate), Punica granatum fruit extract, butylated hydroxytoluene (BHT), L-ergothioneine (available as THIOTANE™), tetrahydrocurcumin, cetyl pyridinium chloride, diethylhexyl syrinylidene malonate (available as OXYNEX™), hexadec-8-ene-1,16-dicarboxylic acid (octadecene dioic acid; ARLATONE™ Dioic DCA from Uniqema), ubiquinone (co-enzyme Q10), yeast extracts or yeast culture fluid (e.g., Piteray), or a combination thereof.

The antioxidant can be a vitamin antioxidant. Non-limiting examples of vitamin antioxidants can include vitamin C, B vitamins including niacinamide, vitamin D, vitamin E, vitamin K, or a combination thereof.

### Reducing Agents

One or more reducing agents can be added to the composition to help mitigate yellowness. The composition can include from about 0.01% to about 0.5% reducing agent, alternatively from about 0.02% to about 0.2%, and alternatively from about 0.05% to about 0.1%.

Non-limiting examples of reducing agents can include sulfite salts, acetyl farnesylcysteine, aminoethanesulfinic acid, butyrolactonethiol, ethanolamine dithiodiglycolate, ethyl thioglycolate, formamidine sulfinic acid, hydrolyzed saccharomyces/lactobacillus/ubiquinone ferment, sodium glyoxylate, sodium hydroxymethane sulfonate, sodium oxymethylene sulfoxylate, sodium thioglycolate, sodium thiosulfate pentahydrate, sodium thiosulfate pentahydrate, potassium sulfite, sodium bisulfite, sodium metabisulfite, sodium thiosulfate, potassium metabisulfite, or a combination thereof.

Non-limiting examples of sulfite salts include sodium sulfite, potassium sulfite, ammonium bisulfite, ammonium sulfite, potassium metabisulfite, potassium sulfite, sodium bisulfite, sodium hydrosulfite, sodium metabisulfite, or a combination thereof.

### Dermatologically Acceptable Carrier

The compositions herein include a dermatologically acceptable carrier (which may be referred to as a “carrier”). The phrase “dermatologically acceptable carrier” means that the carrier is suitable for topical application to the keratinous tissue, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. In some instances, the solubility or dispersibility of the components (e.g., extracts, sunscreen active, additional components) may dictate the form and character of the carrier. Non-limiting examples include simple solutions (e.g., aqueous or anhydrous), dispersions, emulsions, and solid forms (e.g., gels, sticks, flowable solids, or amorphous materials). In some instances, the dermatologically acceptable carrier is in the form of an emulsion that has a continuous aqueous phase (e.g., an oil-in-water or water-in-oil-in-water emulsion) or a continuous oil phase (e.g., water-in-oil or oil-in-water-in-oil emulsion). The oil phase of the emulsion may include silicone oils, non-silicone oils such as hydrocarbon oils, esters, ethers, and mixtures thereof. The aqueous phase may include water and water-soluble ingredients (e.g., water-soluble moisturizing agents, conditioning agents, anti-microbials, humectants and/or other skin care actives). In some instances, the aqueous phase may include components other than water, including but not limited to water-soluble moisturizing agents, conditioning agents, anti-microbials, humectants and/or other water-soluble skin care actives. In some instances, the non-water component of the composition comprises a humectant such as glycerin and/or other polyol(s). The composition can contain from about 1% to about 15%, alternatively from about 3% to about 10%, alternatively from about 4% to about 9%, and alternatively from about 5% to about 8% humectant.

In some instances, the compositions herein are in the form of an oil-in-water (“O/W”) emulsion that provides a sensorial feel that is light and non-greasy. Suitable O/W emulsions herein may include a continuous aqueous phase of more than 50% by weight of the composition, and the remainder being the dispersed oil phase. The aqueous phase may include 1% to 99% water, based on the weight of the aqueous phase, along with any water soluble and/or water miscible ingredients. In these instances, the dispersed oil phase will typically be present at less than 40% by weight of composition (e.g., 1% to 35%, 2% to 30%, 3% to 25%, 4% to 20%, or even 5% to 18%) to help avoid some of the undesirable feel effects of oily compositions. The oil phase may include one or more volatile and/or non-volatile oils (e.g., botanical oils, silicone oils, and/or hydrocarbon oils). Some nonlimiting examples of oils that may be suitable for use in the present compositions are disclosed in U.S. Pat. No. 9,446,265 and U.S. Publication No. 2015/0196464.

The carrier may contain one or more dermatologically acceptable diluents. As used herein, “diluent” refers to materials in which the skin care actives herein can be dispersed, dissolved, or otherwise incorporated. Some non-limiting examples of hydrophilic diluents include water, organic hydrophilic diluents such as lower monovalent alcohols (e.g., C₁-C₄) and low molecular weight glycols and polyols, including propylene glycol, polyethylene glycol (e.g., molecular weight of 200 to 600 g/mole), polypropylene glycol (e.g., Mw of 425 to 2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

### Thickening Agents

The composition may include one or more thickening agents, alternatively at least two thickening agents. In some examples, the composition is a moisturizer and can contain from about 0.5% to about 6% thickening agent, alternatively from about 0.75% to about 5%, and alternatively from about 1% to about 4%. In other examples, the composition is a serum and can contain from about 0.1% to about 4% thickening agent, alternatively from about 0.25% to about 3%, alternatively from about 0.5% to about 2%. Suitable classes of thickening agents include but are not limited to carboxylic acid polymers, polyacrylamide polymers, sulfonated polymers, copolymers thereof, hydrophobically modified derivatives thereof, and mixtures thereof.

The composition can include a carboxylic acid polymer thickening agent such as a carbomer. The composition can contain from about 0.1% to about 1%, alternatively from about 0.2% to about 0.8%, alternatively 0.25% to about 0.7%, alternatively from about 0.3% to about 0.6%, and alternatively from about 0.4% to about 0.5% of a carboxylic acid polymer thickening agent.

The composition can include a polyacrylamide polymer and copolymer thickening agent. The composition can contain from about 0.8% to about 3%, alternatively from about 0.9% to about 2.5%, alternatively from about 1% to about 2.2%, alternatively from about 1.1% to about 2.1%, alternatively from about 1.2% to about 1.9%, or alternatively from about 1.3% to about 1.8% polyacrylamide polymer and copolymer thickening agent.

Suitable thickening agents include carboxylic acid polymers such as the carbomers (e.g., the CARBOPOL® 900 series such as CARBOPOL® 954), and Ultrez 10 and Ultrez 30. Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, Ultrez 20, Ultrez 21, PEMULEN TR-1, and PEMULEN TR-2, from Noveon, Inc.

Other suitable thickening agents include the polyacrylamide polymers and copolymers. An exemplary polyacrylamide polymer has the CTFA designation “polyacrylamide and isoparaffin and laureth-7” and is available under the trade name SEPIGEL 305 from Seppic® Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500 W, SSSA100H, from Lipo Chemicals, Inc., (Patterson, N.J.).

Other suitable thickening agents useful herein are sulfonated polymers such as the CTFA designated sodium polyacryloyldimethyl taurate available under the trade name Simulgel 800 from Seppic® Corp. and Viscolam® at 100 P available from Lamberti S.p.A. (Gallarate, Italy). Another commercially available material comprising a sulfonated polymer is Sepiplus™ 400 available from Seppic® Corp.

Further, suitable thickening agents may include superabsorbent polymers. These superabsorbent polymers may be chosen from: crosslinked sodium polyacrylates, such as, for example, those sold under the names Octacare X100, X110 and RM100 by Avecia®, those sold under the names Flocare GB300 and Flosorb 500 by SNF™, those sold under the names Luquasorb 1003, Luquasorb 1010, Luquasorb 1280 and Luquasorb 1100 by BASF®, those sold under the names Water Lock G400 and G430 (INCI name: Acrylamide/Sodium Acrylate Copolymer) by Grain Processing®, or Aqua Keep® 10 SH NF, Aqua Keep® 10 SH NFC, sodium acrylate crosspolymer-2, provided by Sumitomo Seika, starches grafted by an acrylic polymer (homopolymer or copolymer) and in particular by sodium polyacrylate, such as those sold under the names Sanfresh ST-100C, ST100MC and IM-300MC by Sanyo Chemical Industries®, Makimousse 12 and Makimouse 25 supplied by Kobo Products Inc (INCI name: Sodium Polyacrylate Starch), hydrolysed starches grafted by an acrylic polymer (homopolymer or copolymer), in particular the acryloacrylamide/sodium acrylate copolymer, such as those sold under the names Water Lock A-240, A-180, B-204, D-223, A-100, C-200 and D-223 by Grain Processing® (INCI name: Starch/Acrylamide/Sodium Acrylate Copolymer). Preferred superabsorbent polymers can include Makimousse 12 and Makimousse 25.

Suitable thickening agents for use herein include gums. “Gum” is a broadly defined term in the art. Gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, derivatives thereof and mixtures thereof.

Natural gums are polysaccharides of natural origin, capable of causing a large viscosity increase in solution, even at small concentrations. They can be used as thickening agents, gelling agents, emulsifying agents, and stabilizers. Most often these gums are found in the woody elements of plants or in seed coatings. Natural gums can be classified according to their origin. They can also be classified as uncharged or ionic polymers (polyelectrolytes), examples of which include the following. Natural gums obtained from seaweeds, such as: agar; alginic acid; sodium alginate; and carrageenan. Natural gums obtained from non-marine botanical resources include: gum arabic, from the sap of Acacia trees; gum ghatti, from the sap of Anogeissus trees; gum tragacanth, from the sap of Astragalus shrubs; karaya gum, from the sap of Sterculia trees. Examples of uncharged gums include: guar gum, from guar beans, locust bean gum, from the seeds of the carob tree; beta-glucan, from oat or barley bran; chicle gum, an older base for chewing gum obtained from the chicle tree; dammar gum, from the sap of Dipterocarpaceae trees; glucomannan from the konjac plant; mastic gum, a chewing gum from ancient Greece obtained from the mastic tree; psyllium seed husks, from the Plantago plant; spruce gum, a chewing gum of American Indians obtained from spruce trees; tara gum, from the seeds of the tara tree. Natural gums produced by bacterial fermentation include gellan gum and xanthan gum.

### Dispersed Phase/Oil Phase

### Retinoid

The skin care compositions herein include a safe and effective amount of a retinoid. As used herein, “retinoid” includes all natural and/or synthetic analogs of Vitamin A or retinol or retinol-like compounds which possess the biological activity of Vitamin A in the skin, as well as the geometric isomers and stereoisomers of these compounds. For example, the retinoid may be a retinol ester (e.g., C₂-C₂₂ alkyl esters of retinol, including retinyl palmitate, retinyl acetate, and retinyl propionate), retinol aldehydes, retinal, beta-carotene, and/or retinoic acid (including all-trans retinoic acid and/or 13-cis-retinoic acid). A particularly suitable example of a retinoid for use in the present composition is retinyl propionate (“RP”). These compounds are well known in the art and are commercially available from a number of sources, e.g., Sigma Chemical Company (St. Louis, MO), Boerhinger Mannheim (Indianapolis, IN), BASF (Mt. Olive, NJ), and Roche (Basel, Switzerland). Other suitable retinoids are tocopheryl-retinoate (tocopherol ester of retinoic acid (trans- or cis-), adapalene {6-[3-(1-adamantyl)-4-methoxyphenyl]-2-naphthoic acid}, and tazarotene (ethyl 6-[2-(4,4-dimethylthiochroman-6-yl)-ethynyl]nicotinate). The retinoid may be included as a pure or substantially pure material, or as an extract obtained by suitable physical and/or chemical isolation from natural (e.g., plant) sources.

The present composition may contain 0.0001% to 2% (e.g., 0.005% to 2%, 0.01% to 1%, 0.01% to 0.5%, 0.1% to 0.4%, 0.15% to 0.3%) of the retinoid. In some instances, mixtures of more than one retinoid may be used.

The retinoid present in the skin care compositions herein is stable. In order for the skin care composition to provide the desired health or appearance benefit, it is important to provide a suitable amount of retinoid active.

The composition can have ≥70% RP remaining, alternatively ≥65% RP remaining, alternatively ≥60% RP remaining, alternatively ≥55% RP remaining, alternatively ≥50% RP remaining at any time during the shelf life of the product. The % RP remaining is determined using the HPLC Method, described hereafter. The initial RP is the concentration of RP at the time of manufacture, which can be the % RP claimed on the product packaging or other materials (e.g., website, SmartLabel®, etc.).

### Silicone Oil

The composition can include a silicone oil selected from volatile silicone oil, non-volatile silicone oil, and combinations thereof. The silicone oil can be in the dispersed phase. The composition can include from about 1% to about 35% silicone oil, alternatively from about 5% to about 30% silicone oil, and alternatively from about 10% to about 28% silicone oil.

### Volatile Silicone Oil

Suitable volatile silicones can include cyclic and linear volatile silicones. A description of various volatile silicones is found in Todd, et al. “Volatile Silicone Fluids for Cosmetics”, 91 Cosmetics and Toiletries 27-32 (1976). Suitable cyclic volatile silicones include cyclic dimethyl siloxane chains containing an average of from about 3 to about 5 silicon atoms, preferably from about 4 to about 5 silicon atoms. Exemplary cyclic volatile silicones of varying viscosities include Dow Corning DC 244, DC 245, DC 344, and DC 345; GE Silicones-OSi Specialties Volatile Silicone 7207 and Volatile Silicone 7158; and GE Silicones SF1202. Suitable volatile linear silicones include the polydimethylsiloxanes containing an average of from about 2 to about 8 silicon atoms. Exemplary linear volatile silicones include the Dow Corning DC 200 series with viscosities of 0.65 cst, 1.0 cst, and 2.0 cst. In certain embodiments, the linear volatile silicones generally have viscosities of less than or equal to about 4 centistokes at 25° C., and the cyclic materials generally have viscosities of less than about 6 centistokes at 25° C.

### Non-Volatile Silicone Oils

Suitable non-volatile silicone oils include polysiloxanes. Non-volatile polylsiloxanes may have a viscosity of from about 10 to about 1,000,000 centistokes at 25° C. Such polysiloxanes can be represented by the general chemical formula:

R₃SiO[R₂SiO]ₓSiR₃

wherein each R is independently selected from hydrogen or C1-30 straight or branched chain, saturated or unsaturated alkyl, phenyl or aryl, trialkylsiloxy; and x is an integer from 0 to about 10,000. In certain embodiments, R is methyl or ethyl. Commercially available polysiloxanes include the polydimethylsiloxanes, which are also known as dimethicones, examples of which include the DM-Fluid series from Shin-Etsu, the Vicasil® series sold by Momentive Performance Materials Inc., and the Dow Corning® 200 series sold by Dow Corning Corporation. Specific examples of suitable polydimethylsiloxanes include Dow Corning® 200 fluids (also sold as Xiameter® PMX-200 Silicone Fluids). Suitable dimethicones include those represented by the chemical formula:

R₃SiO[R₂SiO]ₓ[RR′SiO]_{y}SiR₃

wherein R and R′ are each independently hydrogen or C1-30 straight or branched chain, saturated or unsaturated alkyl, aryl, or trialkylsiloxy; and x and y are each integers of 1 to 1,000,000. Examples include alkyl dimethicones wherein at least R′ is a fatty alkyl (e.g., C12-22). A suitable alkyl dimethicone is cetyl dimethicone, wherein R′ is a straight C16 chain and R is methyl, commercially available as 2502Cosmetic Fluid from Dow Corning.

Preferred non-volatile oils include dimethicones (polydimethylsiloxanes), preferably with viscosities of between 10 cst and 1000 cst, more preferably between 15 cst to 400 cst, most preferably between 20 cst and 200 cst. The average chain lengths for these preferred dimethicone materials is from about 12 to about 375 dimethylsiloxane units, more preferably from about 20 to about 200 dimethylsiloxane units, and most preferably with average chain lengths of from about 27 to about 125 dimethylsiloxane units. In one embodiment, the second composition will comprise at least one non-volatile silicone oil. In one such embodiment, at least about 70%, by weight of the non-volatile oil, is a non-volatile silicone oil. In another embodiment, at least about 80%, by weight of the nonvolatile oil, is a non-volatile silicone. In yet another embodiment, at least about 90%, by weight of the non-volatile oil, is a non-volatile silicone oil.

### Other Optional Ingredients

The present composition may optionally include one or more additional ingredients commonly used in cosmetic compositions (e.g., colorants, skin care actives, anti-inflammatory agents, sunscreen agents, emulsifiers, buffers, rheology modifiers, combinations of these and the like), provided that the additional ingredients do not undesirably alter the skin health or appearance benefits provided by the present compositions. The additional ingredients, when incorporated into the composition, should be suitable for use in contact with human skin tissue without undue toxicity, incompatibility, instability, allergic response, and the like. The other optional ingredients can be present in the continuous phase and/or dispersed phase. Some nonlimiting examples of additional actives include vitamins, minerals, peptides and peptide derivatives, sugar amines, sunscreens, oil control agents, particulates, flavonoid compounds, hair growth regulators, anti-oxidants and/or anti-oxidant precursors, preservatives, protease inhibitors, tyrosinase inhibitors, anti-inflammatory agents, moisturizing agents, exfoliating agents, skin lightening agents, sunless tanning agents, lubricants, anti-acne actives, anti-cellulite actives, chelating agents, anti-wrinkle actives, anti-atrophy actives, phytosterols and/or plant hormones, N-acyl amino acid compounds, antimicrobials, and antifungals. In some examples, the composition can include a fragrance, in particular a natural fragrance, or a colorant, in particular a natural colorant. Other non-limiting examples of additional ingredients and/or skin care actives that may be suitable for use herein are described in U.S. Publication Nos. 2002/0022040; 2003/0049212; 2004/0175347; 2006/0275237; 2007/0196344; 2008/0181956; 2008/0206373; 2010/00092408; 2008/0206373; 2010/0239510; 2010/0189669; 2010/0272667; 2011/0262025; 2011/0097286; US2012/0197016; 2012/0128683; 2012/0148515; 2012/0156146; and 2013/0022557; and U.S. Pat. Nos. 5,939,082; 5,872,112; 6,492,326; 6,696,049; 6,524,598; 5,972,359; and 6,174,533.

When including optional ingredients in the compositions herein, it may be desirable to select ingredients that do not form complexes or otherwise undesirably interact with other ingredients in the composition, especially pH sensitive ingredients like niacinamide, salicylates and peptides. When present, the optional ingredients may be included at amounts of from 0.0001% to 50%; from 0.001% to 20%; or even from 0.01% to 10% (e.g., 50%, 40%, 30%, 20%, 10%, 5%, 4%, 3%, 2%, 1%, 0.5% or 0.1%), by weight of the composition.

### Method of Use

The skin care composition may be applied to the face, neck, and/or a portion or combination thereof at least once a day, twice a day, or on a more frequent daily basis, during a treatment period. When applied twice daily, the first and second applications are separated by at least 1 to 12 hours. Typically, the composition is applied in the morning and/or at night before bed. The treatment period herein is ideally of sufficient time for the retinoids and/or other skin care actives to improve the appearance of the skin. The treatment period may last for at least 1 week (e.g., about 2 weeks, 4 weeks, 8 weeks, or even 12 weeks). In some instances, the treatment period will extend over multiple months (i.e., 3-12 months). In some instances, the composition may be applied most days of the week (e.g., at least 4, 5 or 6 days a week), at least once a day or even twice a day during a treatment period of at least 2 weeks, 4 weeks, 8 weeks, or 12 weeks.

The skin care composition can visibly improve fine lines and wrinkles, smoothness, brightness, firming, dark spots, and pores. The skin care composition can leave skin feeling hydrated and replenished. The skin care composition can be applied at night. The skin care composition can even skin texture and minimizes the appearance of pores.

### TEST METHODS

### Color Test Method

For the purposes of the present invention, color is defined according to a value on the CIELAB color system, which is based on XYZ color system, defined by the Commission Internationale de l'Eclairage (CIE system) to provide a manner of objectively representing perceived color and color differences. X, Y, and Z can be expressed in a variety of manners, or “scales,” one of which is the Hunter Scale. The Hunter scale has three variables, L, a, and b, which correlate mathematically to X, Y, and Z, and is described by Robertson, A. R. in “The CIE 1976 Color Difference Formulas,” Color Research Applications, vol. 2, pp. 7-11 (1977).

To measure the color of the compositions of the present invention, approximately 8 mL of the product to be tested is transferred into a petri dish (35×10 mm disposable) using a syringe. The Petri dish is then placed on the black half of a standard opacity chart (Form N2A, Leneta Company of Manwah, NJ or the equivalent thereof, of which the top half is black and the bottom half is white). The color (L, a, and b values) of the product in the petri dish is then measured using an integrated sphere spectrophotometer with settings selected to exclude specular reflection. The value for “a” correlates to a value along the red-green (horizontal) axis, and the value for “b” correlates to a value along the blue-yellow (vertical) axis. For example, a yellow-colored sample will have a positive b-value, whereas a green colored sample will have a negative a-value. A more positive or negative value represents a more intense color. The value for “L” is an indicator of lightness and/or darkness, and correlates to a value along the z-axis, which is perpendicular to both the horizontal and vertical axes.

### HPLC Test Method

This method provides a suitable means for determining the amount of RP loss in a composition.

A sufficient quantity of the test composition is placed in a controlled environment chamber/room at the desired temperature, humidity, and duration. When accelerated aging is complete, the sample is removed from the controlled environment, equilibrated to room temperature (21° C.±2° C.) for 24 hours, and then measured for chemical analysis shortly after. Note that retinoids are light sensitive and should not be exposed to direct light. All test compositions are mixed thoroughly before sampling.

The amount of RP loss is determined on a % w/w basis by HPLC (isocratic elution) as follows. The HPLC column(s) is conditioned in accordance with conventional practices. Chromatographic Conditions
1. Column: C18 (5 micron), 250 mm×4.6 mm
2. Mobile Phase (Eluent): methanol/2-propanol (70/30 v/v)
3. Column temperature: approximately 25° C. (Ambient)
4. UV wavelength: 280 nm
5. Injection volume: 20 microliters
6. Flow rate: 1.0 ml/min
7. Run time: approximately 22.0 minutes
8. RP retention time: 6 minutes

### Mobile Phase Preparation

Prepare 1 L of Mobile Phase (Eluent) by combining 700 mL of methanol with 300 mL of 2-propanol.

External Standard Preparation (Prepare Fresh on Day of Use)

In a 25 ml amber flask, dissolve 25 mg of retinyl propionate in 10 ml of Mobile Phase. Avoid exposing the RP to light. Dilute to volume with Mobile Phase. Transfer a 5.0 ml aliquot to a 50 ml amber flask and dilute to volume with mobile phase. Mix well. Filter about 1 ml of the external standard thru a suitable 0.45 micron filter (e.g., Whatman GD/X) into an amber HPLC sample vial.

### HPLC Sample Preparation

Using a 1-cc tuberculin syringe, weigh and transfer 500 mg of the sample composition into a 25 ml amber volumetric flask. Add 10 mL of Mobile Phase and vortex on high for 2 minutes or until product is completely dispersed. Dilute to volume with mobile phase and mix well. Filter approximately 1 ml into an auto-sampler vial using a syringe filter (e.g., Whatman GD/X filter unit). Perform 20 injections using the condition described.

(A)/(B)×(B)/(W)×(DF)×100=retinyl propionate, % w/w

### Where,

A=peak area of retinyl propionate for the sample
B=peak area of retinyl propionate for the calibration
C=retinyl propionate standard weight in mg
W=sample weight, mg
DF=Dilution Factor (e.g., 0.1)

### EXAMPLES

The following data and examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

Table 5 and Table 6 illustrate examples of the skin care compositions that were made as follows: add the Water Phase ingredients to a suitable mixing container and mix until homogenous. Add the Oil Phase ingredients to a separate container and mix until homogenous. Add the Oil Phase to Water Phase and mill the mixture with a Tekmar® mill TK-25 or equivalent for 2 to 3 mins at 9000-11000 RPM. Replace the mill with a propeller mixer and add the remaining materials (e.g., pH adjuster, feel/appearance modifiers like starch and TiO₂) and/or phases, if present, to the composition one at a time and continue to mix until homogenous. Transfer the batch to final container.

TABLE 5
Examples 1-4

| | Ex. 1 | Ex. 2 | Ex. 3 | Ex. 4 |
|---|---|---|---|---|
| | (wt. %) | (wt. %) | (wt. %) | (wt. %) |
| Water Phase | | | | |
| USP Water | Q.S. | Q.S. | Q.S. | Q.S. |
| Thickeners | 2.4 | 2.4 | 2.4 | 2.4 |
| Water Soluble Actives | 0.13 | 0.13 | 0.13 | 0.13 |
| Humectant | 7.0 | 7.0 | 7.0 | 7.0 |
| Disodium EDTA | 0.050 | 0.050 | 0.050 | 0.050 |
| Phenoxyethanol | | 0.050 | 0.050 | 0.050 |
| Hydroxyacetophenone¹ | | 0.15 | 0.15 | 0.15 |
| DMDM hydantoin and | 0.30 | | | |
| iodopropynyl | | | | |
| butylcarbamate² | | | | |
| Carnosine | | | 0.10 | 0.10 |
| Sodium Sulfite | | | 0.050 | |
| Oil Phase | | | | |
| Silicone Oils | 28 | 28 | 28 | 28 |
| Oil Soluble Actives | 0.0033 | 0.0033 | 0.0033 | 0.0033 |
| Retinyl Propionate | 0.30 | 0.30 | 0.30 | 0.30 |
| Polysorbate 20 | 0.20 | 0.20 | 0.20 | 0.20 |
| Laureth 4 | 0.20 | 0.20 | 0.20 | 0.20 |
| Remaining Materials | | | | |
| Titanium Dioxide, Water, | 0.088 | 0.088 | 0.088 | 0.088 |
| Glycerin, Ammonium | | | | |
| Polyacrylate⁴ | | | | |
| Zea Mays Starch⁵ | 5.0 | 5.0 | 5.0 | 5.0 |
| Aminomethyl Propanol | 0.075 | 0.075 | 0.075 | 0.075 |

¹Symsave ® H from Symrise ®
²Glydant ® Plus Liquid from Arxada ®
⁴GLW75 PFAP-PE from Kobo ® Products Inc.
⁵AMAZE ® HTP from Nouryon ®

TABLE 6
Serum Example 5 and 6

| | Ex. 5 (wt. %) | Ex. 6 (wt. %) |
|---|---|---|
| Water Phase | | |
| USP Water | Q.S. | Q.S. |
| Disodium EDTA | 0.050 | 0.050 |
| Humectant | 3.0 | 3.0 |
| Phenoxyethanol | 0.050 | 0.050 |
| Hydroxyacetophenone¹ | 0.15 | 0.15 |
| Polyacrylate Crosspolymer-6³ | 0.90 | 0.90 |
| Carnosine | | 0.10 |
| Sodium Sulfite | | 0.050 |
| Oil Phase | | |
| Silicone Oils | 14 | 14 |
| Retinyl Propionate | 0.30 | 0.30 |
| PEG-11 Methyl Ether Dimethicone² | 0.30 | 0.30 |

¹Symsave ® H from Symrise ®
²KF-6011 from Shin-Etsu
³Sepimax Zen from Seppic ®

The examples in Table 7 could be made according to the method described herein.

TABLE 7
Examples 7-13

| | Ex. 7 | Ex. 8 | Ex. 9 | Ex. 10 | Ex. 11 | Ex. 12 | Ex. 13 |
|---|---|---|---|---|---|---|---|
| Water Phase | | | | | | | |
| Water | QS | QS | QS | QS | QS | QS | QS |
| Dex-Panthenol | 0.5 | 0.5 | 0.5 | — | — | — | 0.5 |
| Glycerin | 7 | 10 | — | 3 | — | 7 | 15 |
| Butylene Glycol | — | — | 4 | — | — | — | — |
| Disodium EDTA | 0.05 | 0.025 | 0.05 | 0.025 | 0.025 | — | 0.05 |
| Carnosine | 0.1 | — | 0.005 | 0.5 | — | — | — |
| Pomegranate | — | 0.05 | — | — | — | — | — |
| Butylated | — | — | — | — | 0.03 | — | — |
| Hydroxytoluene | | | | | | | |
| Caffeine | — | 0.5 | — | — | — | — | — |
| Tinogard TS¹ | — | — | — | — | — | 0.1 | 0.5 |
| Hydroxyacetophenone | 0.15 | 0.15 | 0.05 | — | 0.15 | — | 0.2 |
| Ethylhexylglycerine | 0.2 | 0.1 | — | — | — | — | — |
| Phenoxyethanol | 0.05 | — | 0.5 | — | 0.15 | — | 0.2 |
| Benzyl Alcohol | — | 0.25 | — | — | — | — | — |
| Symdiol 68² | — | — | — | 0.4 | — | — | — |
| Glycacil L³ | — | — | — | 0.09 | — | — | — |
| Sodium Benzoate | — | — | — | — | 0.05 | — | — |
| Sodium Sulfite | 0.05 | — | 0.1 | — | — | — | — |
| Sodium Metabisulfite | — | 0.2 | — | — | 0.05 | — | — |
| Sodium Bisulfite | — | — | — | 0.05 | — | 0.1 | 0.2 |
| Niacinamide | 2.0 | 2.0 | 5.0 | 3.5 | — | 2.0 | 3.0 |
| Sepiwhite MSH⁴ | — | — | — | — | 1.0 | — | — |
| Glyco-Repair⁵ | — | — | 2.0 | — | 1.0 | — | — |
| Palestrina⁶ | 0.6 | — | 0.4 | — | 1.0 | — | — |
| Olivem 460⁷ | 0.1 | — | — | 0.1 | — | — | — |
| Promatrixyl⁸ | 0.4 | — | 0.7 | 0.05 | — | — | 0.3 |
| Green Tea Extract | — | — | — | — | — | 1.0 | 1.0 |
| pH Adjustor | | | | | | | |
| Triethanolamine | 0.45 | — | 0.1 | — | — | — | — |
| Aminomethyl | — | 0.35 | — | 0.1 | — | 0.1 | — |
| Propanol | | | | | | | |
| Thickener | | | | | | | |
| Sepigel 305⁹ | 0.9 | 1.5 | 2.0 | — | 0.9 | 1.5 | — |
| Simulgel INS-100¹⁰ | — | — | — | 0.9 | — | — | 2.0 |
| Makimousse-12¹¹ | — | — | — | — | 0.5 | — | 0.5 |
| Ultrez-10¹² | 0.2 | — | 0.1 | 0.1 | — | 0.1 | — |
| Ultrez-21¹³ | — | 0.45 | — | 0.1 | — | 0.1 | — |
| Xanthan Gum | — | — | — | — | — | 0.1 | 0.1 |
| Oil Phase | | | | | | | |
| Sodium Hyaluronate | 0.5 | 1.5 | 1.0 | 0.75 | 1.0 | 1.0 | 0.5 |
| Cyclomethicone D5 | 10.0 | 1.0 | 12.0 | — | — | — | 16.0 |
| Dimethicone 2 cst | 5.0 | — | 2.0 | 12.0 | — | 6.0 | — |
| Dimethicone 5 cst | — | 16.0 | — | 3.0 | 14.0 | 9.0 | 2.0 |
| Dimethicone 50 cst | — | — | — | 2.0 | 3.0 | 2.0 | — |
| Dimethicone 350 cst | — | — | 1.0 | — | — | — | — |
| DC9041¹⁴ | 0.5 | — | — | 1.0 | — | — | 2.0 |
| Hexyldecanol | — | — | — | 0.1 | 5.0 | — | — |
| Retinyl Propionate | 0.3 | 0.15 | 0.45 | 0.1 | 0.3 | — | 0.3 |
| Retinol 15 D¹⁵ | — | 0.05 | — | — | — | 1 | — |
| Laureth-4 | 0.1 | 0.2 | 0.3 | — | 0.2 | 0.1 | 0.3 |
| DC1503¹⁶ | — | 2.0 | — | — | 1.5 | — | — |
| Polysorbate 20 | 0.1 | 0.2 | — | 0.3 | 0.2 | 0.2 | — |

¹Octadecyl Di-t-butyl-4-hydroxyhydrocinnamate, from BASF ®
²1,2-hexanediol and caprylyl glycol, from Symrise ®
³Iodopropynyl butylcarbamate, PEG-4 laurate, PEG-4 dilaurate, and polyethylene glycol, from Arxada ®
⁴Undecylenoyl phenylalanine, from Seppic ® Corporation
⁵Water and hydrolyzed ceratonia siliqua seed extract, from Silab ®
⁶Water, glycerin, decyl glucoside, lactic acid, benzyl alcohol, and palmitoyl dipeptide-7, from Sederma ®
⁷Sodium PEG-7 olive oil carboxylate, from B&T S.r.l.
⁸Water, glycerin, PEG-100 stearate, benzyl alcohol, and palmitoyl pentapeptide-4, from Sederma
⁹Polyacrylamide, C13-14 Isoparaffin, and laureth-7, from Seppic ® Corporation
¹⁰Hydroxyethyl Acrylate/Sodium Acryloyldimethyl Taurate Copolymer (and) Isohexadecane (and) Polysorbate 60, form Seppic ® Corporation
¹¹Sodium polyacrylate starch, from Kobo ® Products Inc.
¹²Carbomer, from Lubrizol ®
¹³Acrylates C10-30 alkyl acrylate crosspolymer, from Lubrizol ®
¹⁴Dimethicone (and) dimethicone crosspolymer, from Dow Corning ®
¹⁵Retinol (and) Caprylic/Capric Triglyceride, from BASF ®
¹⁶Dimethicone (and) dimethiconol, from Dow Corning ®

### Combinations:

A. A skin care composition comprising:
a. a continuous phase comprising:
   i. from about 0.01% to about 1.5% of a preservative system, preferably about 25 comprising 0.1% to about 1% preservative system, and more preferably from about 0.15% to about 0.5% preservative system, wherein the preservative system comprises one or more cell wall disrupters;
   ii. from about 0.01% to about 1% of one or more antioxidants, preferably about 0.05% to about 0.75% of one or more antioxidants, and more preferably from about 0.1% to about 0.3% of one or more antioxidants;
   iii. from about 0.01% to about 0.5% of one or more reducing agents, preferably from about 0.02% to about 0.2% of one or more reducing agents, and more preferably from about 0.05% to about 0.1% of the one or more reducing agents;
   iv. water;
b. a dispersed phase comprising from about 0.01% to about 1% of one or more retinoid, preferably from about 0.01% to about 0.5%, more preferably from about 0.1% to about 0.4%;
wherein the composition is free of DMDM hydantoin, imadazolidinyl urea, diazlidinyl urea, sodium hydroxyl, and methyl glycinate.

B. The skin care composition according to Paragraph A, wherein the one or more retinoids comprises retinyl palmitate, retinyl acetate, retinyl propionate, or a combination thereof.

C. The skin care composition according to Paragraphs A-B, wherein the cell wall disrupter is selected from the group consisting of aromatic alcohols, mid chain diols, organic acids, hydroxyacetophenone, 1,2-Hexanediol (and) Caprylyl Glycol, ethylhexylglycerin, methylheptylglycerin, or a combination thereof.

D. The skin care composition according to Paragraphs A-C, wherein the cell wall disrupter comprises hydroxyacetophenone and an aromatic alcohol selected from the group consisting of phenoxyethanol, tryptophol, tyrosol, phenethyl alcohol, benzyl alcohol, or a combination thereof.

E. The skin care composition according to Paragraph D, wherein the aromatic alcohol comprises phenoxyethanol.

F. The skin care composition according to Paragraphs A-E, wherein the antioxidant comprises a non-vitamin antioxidant comprising hydroxycinnamic acid, ferulic acid, bakuchiol, tea extracts, caffeine, Rosmarinus officinalis leaf extract, Octadecyl Di-t-butyl-4-hydroxyhydrocinnamate, carnosine, Ethylene bis(oxyethylene) bis-(3-(5-tert-butyl-4-hydroxy-m-tolyl)propionate), Punica granatum fruit extract, butylated hydroxytoluene, L-ergothioneine, tetrahydrocurcumin, cetyl pyridinium chloride, diethylhexyl syrinylidene malonate, hexadec-8-ene-1,16-dicarboxylic acid, ubiquinone, yeast extracts, yeast culture fluid, or a combination thereof.

G. The skin care composition according to Paragraphs A-F, wherein the antioxidant comprises carnosine.

H. The skin care composition according to Paragraphs A-G, wherein the reducing agent comprises sulfite salts, acetyl farnesylcysteine, aminoethanesulfinic acid, butyrolactonethiol, ethanolamine dithiodiglycolate, ethyl thioglycolate, formamidine sulfinic acid, hydrolyzed saccharomyces/lactobacillus/ubiquinone ferment, sodium glyoxylate, sodium hydroxymethane sulfonate, sodium oxymethylene sulfoxylate, sodium thioglycolate, sodium thiosulfate pentahydrate, sodium thiosulfate pentahydrate, potassium sulfite, sodium bisulfite, sodium metabisulfite, sodium thiosulfate, potassium metabisulfite, or a combination thereof.

I. The skin care composition according to Paragraphs A-H, wherein the reducing agent comprises a sulfite salt comprising sodium sulfite, potassium sulfite, ammonium bisulfite, ammonium sulfite, potassium metabisulfite, potassium sulfite, sodium bisulfite, sodium hydrosulfite, sodium metabisulfite, or a combination thereof.

J. The skin care composition according to Paragraphs A-I, further comprising one or more chelating agents comprising disodium EDTA, tetrasodium EDTA, tetrahydroxypropyl ethylenediamine, etidronic acid, sodium phytate, phytic acid, oxalic acid and derivatives, sodium gluconate, trisodium ethylenediamine disuccinate, or a combination thereof.

K. The skin care composition according to Paragraphs A-J, wherein the chelant comprises disodium EDTA.

L. The skin care composition according to Paragraphs A-K, wherein the composition comprises a b-value of less than 25, preferably less than 20, and more preferably less than 15, according to the Color Test Method.

M. The skin care composition according to Paragraphs A-L, wherein the composition comprises retinyl propionate and the skin care composition comprises greater than or equal to 50% of the retinyl propionate remaining as compared to the amount of retinyl propionate at the time of manufacture, preferably greater than or equal to 65% of the retinyl propionate remaining, and more preferably greater than or equal to 70% of the retinyl propionate remaining, according to the HPLC Test Method.

N. The skin care composition according to Paragraphs A-M, wherein the skin care composition is free of formaldehyde and formaldehyde-releasing agents.

O. The skin care composition according to Paragraphs A-N, wherein the skin care composition is free of parabens and sulfate-based surfactants.

P. The skin care composition according to Paragraphs A-O, wherein the dispersed phase further comprises silicone.

Q. The skin care composition according to Paragraphs A-P, wherein the dispersed phase further comprises from about 1% to about 35% silicone oil, preferably from about 5% to about 30% silicone oil, and more preferably from about 10% to about 28% silicone oil.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition comprising:
a. a continuous phase comprising:
i. a preservative system comprising hydroxyacetophenone;
ii. carnosine;
iii. sodium sulfite;
iv. water;
b. a dispersed phase comprising retinyl propionate;
wherein the composition is free of DMDM hydantoin, imadazolidinyl urea, diazlidinyl urea, sodium hydroxyl, and methyl glycinate.

2. The skin care composition of claim 1, wherein the preservative system further comprises phenoxyethanol.

3. The skin care composition of claim 1, further comprising one or more chelating agents selected from disodium EDTA, tetrasodium EDTA, tetrahydroxypropyl ethylenediamine, etidronic acid, sodium phytate, phytic acid, oxalic acid and derivatives, sodium gluconate, trisodium ethylenediamine disuccinate, or mixtures thereof.

4. The skin care composition of claim 3, wherein the chelant comprises disodium EDTA.

5. The skin care composition of claim 1, wherein the composition comprises a b-value of less than 25, according to the Color Test Method.

6. The skin care composition of claim 5, wherein the composition comprises a b-value of less than 20 according to the Color Test Method.

7. The skin care composition of claim 1, wherein the composition comprises greater than or equal to 50% of the retinyl propionate remaining as compared to the amount of retinyl propionate at the time of manufacture.

8. The skin care composition of claim 7, wherein the composition comprises greater than or equal to 65% of the retinyl propionate remaining as compared to the amount of retinyl propionate at the time of manufacture.

9. The skin care composition of claim 1, wherein the skin care composition is free of formaldehyde and formaldehyde-releasing agents.

10. The skin care composition of claim 1, wherein the skin care composition is free of parabens and sulfate-based surfactants.

11. The skin care composition of claim 1, wherein the dispersed phase further comprises silicone.

12. A skin care composition comprising:
a. a continuous phase comprising:
i. from about 0.1% to about 1% of a preservative system comprising one or more cell wall disrupters;
ii. from about 0.01% to about 1% of one or more antioxidants;
iii. from about 0.02% to about 0.25% of one or more reducing agents;
iv. water;
b. a dispersed phase comprising from about 0.01% to about 2% of one or more retinoid;
wherein the skin care composition is free of formaldehyde and formaldehyde-releasing agents.

13. The skin care composition of claim 12, wherein the one or more retinoids is selected from retinyl palmitate, retinyl acetate, retinyl propionate, or mixtures thereof.

14. The skin care composition of claim 12, wherein cell wall disrupter is chosen from the group consisting of aromatic alcohols, mid chain diols, organic acids, hydroxyacetophenone, 1,2-Hexanediol (and) Caprylyl Glycol, ethylhexylglycerin, methylheptylglycerin, or mixtures thereof.

15. The skin care composition of claim 12, wherein the antioxidant is chosen from hydroxycinnamic acid, ferulic acid, bakuchiol, tea extracts, caffeine, Rosmarinus officinalis leaf extract, Octadecyl Di-t-butyl-4-hydroxyhydrocinnamate, carnosine, Ethylene bis(oxyethylene) bis-(3-(5-tert-butyl-4-hydroxy-m-tolyl)propionate), Punica granatum fruit extract, butylated hydroxytoluene, L-ergothioneine, tetrahydrocurcumin, cetyl pyridinium chloride, diethylhexyl syrinylidene malonate, hexadec-8-ene-1,16-dicarboxylic acid, ubiquinone, yeast extracts, yeast culture fluid, or mixtures thereof.

16. The skin care composition of claim 12, wherein the reducing agent is selected from sulfite salts, acetyl farnesylcysteine, aminoethanesulfinic acid, butyrolactonethiol, ethanolamine dithiodiglycolate, ethyl thioglycolate, formamidine sulfinic acid, hydrolyzed saccharomyces/lactobacillus/ubiquinone ferment, sodium glyoxylate, sodium hydroxymethane sulfonate, sodium oxymethylene sulfoxylate, sodium thioglycolate, sodium thiosulfate pentahydrate, sodium thiosulfate pentahydrate, potassium sulfite, sodium bisulfite, sodium metabisulfite, sodium thiosulfate, potassium metabisulfite, or mixtures thereof.

17. The skin care composition of claim 16, wherein the reducing agent comprises a sulfite salt chosen from sodium sulfite, potassium sulfite, ammonium bisulfite, ammonium sulfite, potassium metabisulfite, potassium sulfite, sodium bisulfite, sodium hydrosulfite, sodium metabisulfite, or mixtures thereof.

18. The skin care composition of claim 12, wherein the dispersed phase further comprises from about 5% to about 30% silicone oil.
